(19) United States
(12) Patent Application Publication
(10) Pub. No.: US 2002/0116063 A1
(43) Pub. Date: Aug. 22, 2002

(54) Kit for chondrocyte cell transplantation

(76) Inventors: Bruno Giannetti, Bonn (DE); Samuel S. Asculai, Toronto (CA)

Correspondence Address: MORGAN, LEWIS & BOCKIUS LLP, 1701 MARKET STREET, PHILADELPHIA, PA, 19103-2921, US

(21) Appl. No.: 10/061,655

(22) Filed: Feb. 1, 2002

Related U.S. Application Data
(63) Continuation of application No. PCT/IB00/01093, filed on Aug. 2, 2000.
(60) Provisional application No. 60/146,683, filed on Aug. 2, 1999.

(30) Foreign Application Priority Data
Feb. 8, 2001 (WO) 01/08610

(51) Int. Cl. A61F 2/18

(52) U.S. Cl. 623/10

(57) ABSTRACT

A kit and a method for the effective treatment of articulating joint surface cartilage (10) by the transplantation of chondrocytes (18) to a surface to be treated. The method includes the steps of placing chondrocytes (18) in a defect (30) of the articulating joint surface, and covering the surface to be treated with an absorbable covering cap (16). The present invention also includes a kit for chondrocyte transplantation including a covering cap (16) and securing device.

### BACKGROUND OF THE INVENTION

The invention relates to improvements in methods of chondrocyte cell transplantation.

U.S. Pat. No. 5,759,190, hereby incorporated by reference, describes one method for transplantation for effective chondrocyte and/or cartilage transplantation. U.S. Provisional Patent Application No. 60/096,597, also hereby incorporated by reference, describes a second method for effective chondrocyte and/or cartilage transplantation. Brittberg et al., Treatment of Deep Cartilage Defects in the Knee with Autologous Chondrocyte Transplantation, New England Journal of Medicine, 331: 889-895 (Oct. 6, 1994), also hereby incorporated by reference, describes a third method of chondrocyte transplantation. U.S. pat. application Ser. No. 09/373,952, also hereby incorporated by reference, describes methods for effective chondrocyte cell and or cartilage transplantation.

Heretofore, it was thought that successful chondrocyte cell and/or cartilage cell transplantation required removal of damaged cartilage down to the underlying bone.

### BRIEF SUMMARY OF THE INVENTION

The present invention includes a system for implanting chondrocyte cells and/or cartilage cells at a site of cartilage damage. The invention involves first removing damaged cartilage from a site of damaged cartilage such that the depth of removal of the cartilage is sufficient to preserve a layer of protective covering, sometimes referred to as a subchondral layer, over the bone. One way of protecting the subchondral layer is to remove the damaged cartilage such that a thin layer of cartilage is left over the subchondral layer. The chondrocyte cells are then transplanted on top of this thin cartilage layer. Leaving a thin layer of cartilage over the subchondral layer limits or entirely prevents bleeding from the site of damaged cartilage.

In this way, chondrocyte cells and/or cartilage cells are implanted by various methods without the use of a hemostatic barrier in the site of damage, as was previously thought necessary.

In one embodiment, the present invention includes a method for the effective treatment of articulating joint surface cartilage by the transplantation of chondrocytes, to a surface to be treated. The method includes the steps of placing chondrocytes in a defect of the articulating joint surface, and covering the surface to be treated with an absorbable covering cap. The present invention also includes a kit for chondrocyte transplantation including a covering cap and securing device.

### BRIEF DESCRIPTION OF THE DRAWINGS

The present invention may be better understood by reference to the description which follows taken together with the accompanying figures which illustrate particular embodiments the present invention wherein:

FIG. 1 is a drawing showing one embodiment of implantation of chondrocyte cells and/or cartilage cells at a site of cartilage damage where the damaged cartilage is removed to a depth above the subchondral layer.

FIG. 2 is a drawing showing a second embodiment of implantation of chondrocyte cells and/or cartilage cells at a site of cartilage damage where the damaged cartilage is removed to a depth above the subchondral layer.

FIG. 3 is a drawing showing a third embodiment of implantation of chondrocyte cells and/or cartilage cells at a site of cartilage damage where the damaged cartilage is removed to a depth above the subchondral layer.

FIG. 4 is a drawing showing a covering cap or matrix used in the methods according to the present invention.

### DETAILED DESCRIPTION OF THE INVENTION

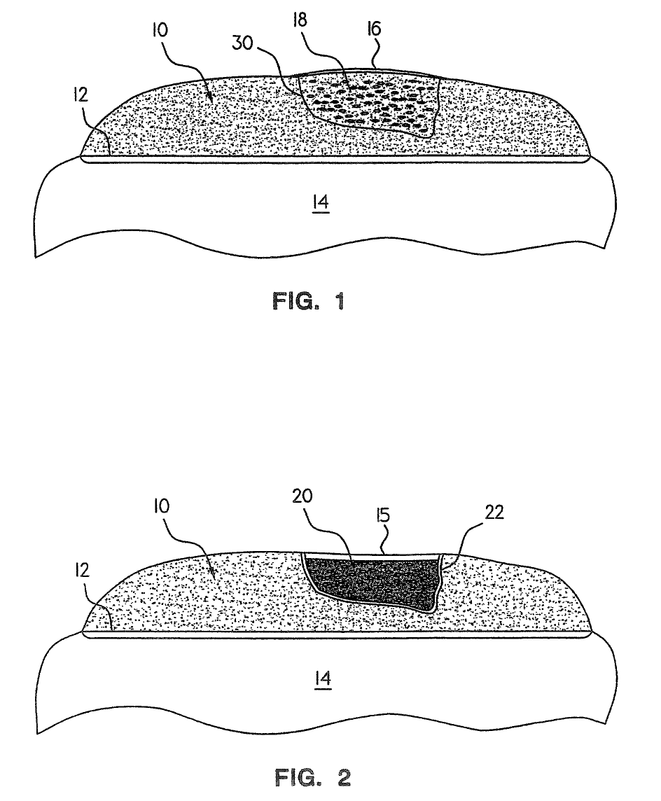

This invention concerns transplantation of chondrocyte cells and/or cartilage cells into a site of cartilage damage without the use of a hemostatic barrier. FIG. 1 shows a first embodiment where damaged cartilage 10 (damaged either through traumatic injury or otherwise defective) is removed to a depth above a subchondral layer 12 covering a bone 14. The thickness of the remaining cartilage layer over the subchondral layer will vary on the site of damage, but is thick enough to prevent or limit the amount of bleeding at the site of damage.

In one embodiment, the present invention includes a cartilage repair implantation method. The implantation method includes harvesting cartilage cells from a non-weight bearing surface of a patient, culturing the chondrocyte cells in a suitable growth media, securing a covering cap 16 over the cartilage defect area leaving one edge of covering cap 16 unsecured, injecting the cultured chondrocytes in growth media under covering cap 16, and securing the open edge of covering cap 16 to the edge of the cartilage defect.

In one embodiment, covering cap 16 preferably is a cell free cap 16 and is used as a patch to cover the damaged area and under which cultured chondrocyte cells such as autologous or homologous chondrocyte cells are transplanted. Covering cap 16 is sutured or otherwise held in place over the area of defect. Covering cap 16 is formed, for example, of a collagen membrane such as Chondro-Cell® (a commercially available Type II collagen matrix pad, Ed. Geistlich Söhne, Switzerland) or Chondro-Gide® (a commercially available Type I collagen matrix pad, Ed. Geistlich Söhne, Switzerland), or any other suitable membrane that will be absorbed or resorbed by the body, as discussed below. The cultured chondrocyte cells in a suitable transplant media 18 are injected under covering cap 16. Transplant media 18, for example, includes DMEM/F12 media (up to 250 ml), autologous serum (25 ml to a final concentration of 10%), L-ascorbic acid (7.5 ml at concentration of 300 micromoles per liter), Fungizone® (2 ml at concentration of 2.2 micromoles per liter), and gentomycin sulfate (1.25 ml at concentration of 70 micromoles per liter). The cultured chondrocyte cells were previously grown in a culture media, for example, including DMEM/F12 media (up to 500 ml), L-ascorbic acid (15 ml at concentration of 300 micromoles per liter), Fungizone® (4.0 ml at concentration of 2.2 micromoles per liter), gentomycin sulfate (2.5 ml at concentration of 70 micromoles per liter), and fetal calf, porcine, kangaroo, or other blood serum (100 ml to final concentration of 20%). Preferably, the cultured chondrocyte cells are moved from the growth media into the transplant media approximately 72 hours before transplantation. By cell-free is meant that covering cap 16 contains no living or dead cells.

In one embodiment, the present invention is as follows. For an autologous implant, a cartilage biopsy is harvested by arthroscopic technique from a non-weight bearing area in a joint of a patient and transported to a laboratory in a growth media containing 20% fetal calf serum. The cartilage biopsy is then treated with an enzyme such as typsin ethylenediaminetetraacetic acid (EDTA), a proteolytic enzyme and binding agent, to isolate and extract cartilage chondrocyte cells. The extracted chondrocyte cells are then cultured in the growth media from an initial cell count of about 50,000 cells to a final count of about 20 million chondrocyte cells or more.

Three (3) days before reimplantation, the growth media is exchanged for a transplant media which contains 10% autologous serum (that is, serum extracted from the patient's blood as described below). Then, the cultured chondrocyte cells in the transplant media are injected under partially secured covering cap 16.

It is understood that the area of cartilage defect 30 can be treated directly, enlarged slightly, or sculpted by surgical procedure prior to injection of cultured chondrocyte cells (as described in U.S. Pat. No. 5,989,269, the entire disclosure and teachings of which are hereby incorporated by reference), to accommodate and promote cartilage cell growth. The culturing procedure as well as the growth and transplant media are described by way of example, in detail below, starting first with a description of a laboratory procedure used to process the harvested cartilage biopsy and to culture the chondrocyte cells according to the present invention.

In one embodiment, growth media herein, “the growth media”) used to treat the cartilage biopsy during the culturing process and to grow the cartilage chondrocyte cells is prepared by mixing together 2.5 ml gentomycin sulfate (concentration 70 micromole/liter), 4.0 ml amphotericin (concentration 2.2 micromole/liter; tradename Fungizone®, an antifungal available from Squibb), 15 ml l-ascorbic acid (300 micromole/liter), 100 ml fetal calf serum (final concentration 20%), and the remainder DMEM/F12 media to produce about 400 ml of growth media. (The same growth media is also used to transport the cartilage biopsy from the hospital to the laboratory in which the chondrocyte cells are extracted and multiplied.)

Blood obtained from the patient is centrifuged at approximately 3,000 rpm to separate the blood serum from other blood constituents. The separated blood serum is saved and used at a later stage of the culturing process and transplant procedure.

Cartilage biopsy previously harvested from a patient for autologous transplantation is shipped in the growth media described above to the laboratory where it will be cultured. The growth media is decanted to separate out the cartilage biopsy, and discarded upon arrival at the laboratory. The cartilage biopsy is then washed in plain DMEM/F12 at least three times to remove the film of fetal calf serum on the cartilage biopsy.

The cartilage biopsy is then washed in a composition which includes the growth media described above, to which 28 ml trypsin EDTA (concentration 0.055) has been added. In this composition it is incubated for five to ten minutes at 37° C., and 5% CO₂. After incubation, the cartilage biopsy is washed two to three times in the growth media, to cleanse the biopsy of any of the typsin enzyme. The cartilage is then weighed. Typically, the minimum amount of cartilage required to grow cartilage chondrocyte cells is about 80-100 mg. A somewhat larger amount, such as 200 to 300 mg, is preferred. After weighing, the cartilage is placed in a mixture of 2 ml collagenase (concentration 5,000 enzymatic units; a digestive enzyme) in approximately 50 ml plain DMEM/F12 media, and minced to allow the enzyme to partially digest the cartilage. After mincing, the minced cartilage is transferred into a bottle using a funnel, and approximately 50 ml of the collagenase and plain DMEM/F12 mixture is added to the bottle. The minced cartilage is then incubated for 17 to 21 hours at 37° C., and 5% CO₂.

In one embodiment, the incubated minced cartilage is then strained using 40 μm mesh, centrifuged (at 1054 rpm, or 200 times gravity) for 10 minutes, and washed twice with growth media. The chondrocyte cells are then counted to determine their viability, following which the chondrocyte cells are incubated in the growth media for at least two weeks at 37° C., and 5% CO₂, during which time the growth media is changed, preferably, three or four times.

Preferably, at least three days before re-implantation in the patient, the chondrocyte cells are removed by trypsinization and centrifugation from the growth media, and transferred to a transplant media containing 1.25 ml gentomycin sulfate (concentration 70 micromole/liter), 2.0 ml amphotericin (concentration 2.2 micromole/liter; tradename Fungizone®, an antifungal available from Squibb), 7.5 ml l-ascorbic acid (300 micromole/liter), 25 ml autologous blood serum (final concentration 10%), and the remainder DMEM/F12 media to produce about 300 ml of transplant media.

Before or during the chondrocyte transplantation procedure, covering cap 16 is cut to a size suitable to fit over the damaged cartilage area. Covering cap 16 is secured by adhesive or mechanical retention devices or means, or a combination of both adhesive or mechanical retention devices or means, to the cartilage defect area without impairing the further in situ differentiation of the chondrocytes and the regeneration of the natural cartilage matrix material. For example, covering cap 16 is sutured, adhered with adhesive, and/or secured with retention pins to the area of cartilage defect 30.

In one embodiment, using a 1 ml syringe and a 16 gauge needle, the cultured chondrocyte cells in transplant media (about 0.6 ml containing about 10×10⁶ chondrocyte cells) was drawn up into the barrel of the syringe. A 23 gauge short needle was switched for the 16 gauge needle and the cultured chondrocyte cells were injected under the secured covering cap 16 into the area of cartilage defect 30. The unsecured opening of covering cap 16 was then secured (for example, with adhesive) prior to removal of the needle and then the needle was carefully withdrawn. No leakage of cells occurred.

Suitable adhesive includes a biocompatible glue, such as organic fibrin glue (e.g., Tisseel®, fibrin based adhesive, Baxter, Austria or a fibrin glue prepared in the surgical theater using autologous blood samples).

FIG. 2 shows a second embodiment where damaged cartilage 10 is removed to a depth above a subchondral layer 12 covering a bone 14. Chondrocyte cells are previously grown on a matrix 15 formed, for example, of a collagen membrane such as Chondro-Cell® (a commercially available Type II collagen matrix pad, Ed. Geistlich Söhne, Switzerland) or Chondro-Gide® (a commercially available Type I collagen matrix pad, Ed. Geistlich Söhne, Switzerland), or any other suitable membrane that will be absorbed or resorbed by the body to form a chondrocyte cell-loaded matrix 20. The chondrocyte cell-loaded matrix 20 is then glued, for example, using a biocompatible glue 22 such as Tisseal® (a commercially available fibrin based adhesive, Baxter, Austria) into the area of damaged cartilage. The cultured chondrocyte cells were previously grown on the matrix 15 in a culture media, for example, including DMEM/F12 media (up to 500 ml), Lascorbic acid (15 ml at concentration of 300 micromoles per liter), Fungizone ® (4.0 ml at concentration of 2.2 micromoles per liter), gentomycin sulfate (2.5 ml at concentration of 70 micromoles per liter), and fetal calf, porcine, kangaroo, or other blood serum (100 ml to final concentration of 20%). At some point after the chondrocyte cells are grown on the matrix but before transplantation, the growth media is exchanged for a transplant media including DMEM/F12 media (up to 250 ml), autologous serum (25 ml to a final concentration of 10%), L-ascorbic acid (7.5 ml at concentration of 300 micromoles per liter), Fungizone® (2 ml at concentration of 2.2 micromoles per liter), and gentomycin sulfate (1.25 ml at concentration of 70 micromoles per liter). The exchange of transplant media for growth media takes place approximately 72 hours before transplantation.

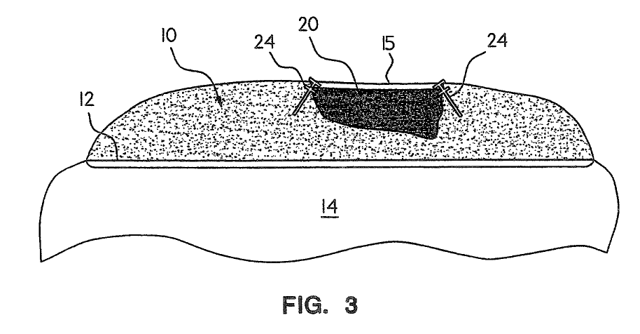

FIG. 3 shows a third embodiment which is identical to the embodiment in FIG. 2 but uses pins 24 to hold the chondrocyte cell-loaded matrix in place rather than fibrin glue. Pins 24 are a commercially available lactide co-polymer pin, sold under the name OrthoPin™ and available from Ed. Geistlich Söhne, Switzerland.

Preferably, covering cap 16 or matrix 15 is a material which will support chondrocyte cell growth and which, over time will be absorbed or resorbed in a body of a patient receiving the implant. The transplantation procedure may be by arthroscopic, minimally invasive or open surgery technique. The method of the invention also contemplates the use of suitable allogenic and xenogenic chondrocyte cells for the repair of a cartilage defect.

A suitable covering cap 16 or matrix 15 is a solid or gel-like, scaffold characterized by being able to hold a stable form for a period of time to enable it to be secured over or in the cartilage defect and to promote growth of chondrocytes cells in the cartilage defect.

Covering cap 16 or matrix 15 is stable for a period of time sufficient to allow full cartilage repair and then be absorbed or resorbed by the body over time, for example, within two to three months from implantation without leaving any significant traces and without forming toxic degradation products. The terms “absorbed” and “resorbed” are meant to include processes by which covering cap 16 or matrix 15 is broken down by natural biological processes, and the broken down covering cap 16 or matrix 15 and degradation products thereof are taken up and disposed of, for example, in cells, across tissues or by way of diffusion or osmosis, through such systems as the lymphatics or blood vessels. Accordingly, covering cap 16 or matrix 15 preferably is a physiologically absorbable or resorbable, non-antigenic membrane-like material.

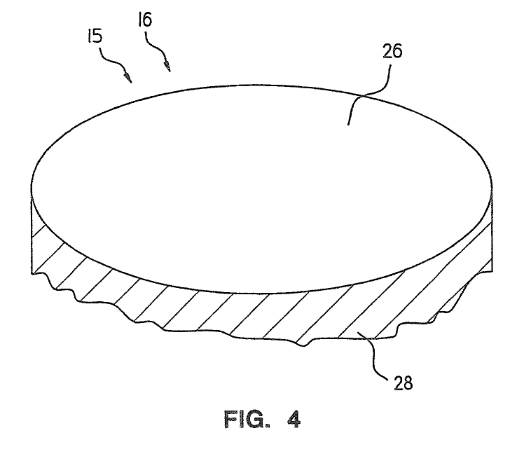

As shown in FIG. 4 covering cap 16 or matrix 15 preferably is in a sheet like form having one relatively smooth side 26 and one relatively rough or porous side 28. Smooth side 26 is relatively more dense than rough or porous side 28. Rough side 28, for example, is fibrous and typically faces cartilage defect 30 and promotes chondrocyte cell ingrowth, while the smooth side 26 typically faces away from cartilage defect 30 and impedes tissue ingrowth.

In one embodiment, covering cap 16 or matrix 15 is formed of polypeptides or proteins. Preferably, the polypeptides or proteins are obtained from natural sources, e.g., from mammals. Artificial materials, however, having physical and chemical properties comparable to polypeptides or proteins from natural sources, may also be used to form covering cap 16 or matrix 15. In another embodiment, covering cap 16 or matrix 15 is formed from hyaluronic acid or derivatives thereof.

It is also preferred that covering cap 16 or matrix 15 is reversibly deformable without mechanical destruction as it is handled by the user so it can be manipulated and then returns to its original shape. This deformation is completely reversible once covering cap 16 or matrix 15 is introduced into the joint or is placed on the surface to be treated, for example, in an arthroscopic procedure.

The material forming covering cap 16 or matrix 15 may be uncrosslinked or partially or fully crosslinked. A preferred material from which covering cap 16 or matrix 15 is formed is collagen such as obtained from equine, porcine, fetal calf, kangaroo, bovine, ovine, and chicken. As set forth above, suitable materials from which covering cap 16 or matrix 15 is formed include Chondro-Cell® (a commercially available type II collagen matrix pad, Ed. Geistlich Söhne, Switzerland), and Chondro-Gide® (a commercially available type I collagen matrix pad, Ed. Geistlich Söhne, Switzerland), as discussed above. A covering cap 16 or matrix 15 formed of collagen Type I is somewhat stiffer than one formed from collagen Type II, although Type II collagen matrixes may also be used as covering cap 16 or matrix 15 in the present invention.

It has been found under electron microscopy that the chondrocytes cultured on the dense or smooth side 26 of covering cap 16 or matrix 15 did not grow into the structure of covering cap 16 or matrix 15, whereas chondrocyte cells cultured on rough or porous side 28 of covering cap 16 did grow into the porous or rough side 28 of covering cap 16 or matrix 15, and furthermore showed the presence of proteoglycans and no signs of fibroblast structures. This result indicates that when covering cap 16 or matrix 15 is secured over or in the area cartilage defect 30, the rough or porous side should face toward the area of damaged cartilage. This will enable the chondrocyte cells to penetrate the rough or porous side of covering cap 16 or matrix 15 and produce a smooth cartilage surface in line with the intact surface of the damaged cartilage.

The present invention encompasses still other methods of cell transplantation so long as such methods avoid the prior necessity of using a hemostatic barrier.

The subjoined claims therefore are intended to be construed to cover not only those embodiments of this invention disclosed above but also to cover all such embodiments, variants and equivalents of the invention as may be made by those skilled in the art to which the invention pertains, which embodiments, variants and equivalents are within the true spirit and scope of this invention.

What is claimed:

1. A method for the effective treatment of articulating joint surface cartilage by the transplantation of chondrocytes, to a surface to be treated, the method comprising the steps:
(a) removing cartilage from the surface to be treated;
(b) placing chondrocytes upon the surface to be treated; and
(c) covering the surface to be treated with an absorbable covering cap.

2. A method according to claim 1, wherein the covering cap has a porous surface.

3. A method according to claim 2, wherein the porous surface of the covering cap is directed toward the surface to be treated.

4. A method according to claim 1, wherein the covering cap is collagen.

5. A method according to claim 1, wherein the covering cap contains hyaluronic acid.

6. A method according to claim 1, wherein the covering cap is cell free.

7. A method for the effective treatment of articulating joint surface cartilage by the transplantation of chondrocytes, to a surface to be treated, the method comprising the steps:
(a) placing chondrocytes in a defect of the articulating joint surface; and
(b) covering the surface to be treated with an absorbable covering cap.

8. The method according to claim 7, wherein the covering cap has a porous surface.

9. The method according to claim 8, wherein the porous surface of the covering cap is directed toward the surface to be treated.

10. The method according to claim 7, wherein the covering cap is collagen.

11. A method according to claim 7, wherein the covering cap is cell free.

12. The method according to claim 7, wherein the covering cap contains hyaluronic acid.

13. The method according to claim 7, wherein the covering cap is partially attached to the surface to be treated prior to placing of the chondrocytes on the surface to be treated in said step (b).

14. A kit for chondrocyte transplantation comprising a covering-cap and a securing device.

15. The kit according to claim 14, wherein the securing device is an adhesive.

16. The kit according to claim 14, wherein the securing device is organic glue.
